(19) United States
(12) Patent Application Publication
(10) Pub. No.: US 2004/0216178 A1
(43) Pub. Date: Oct. 28, 2004

(54) Regulation of mdm2 function

(76) Inventors: Stephen N. Jones, Shrewsbury, MA (US); Heather Steinman, North Grafton, MA (US)

Correspondence Address: FISH & RICHARDSON PC, 225 FRANKLIN ST, BOSTON, MA, 02110, US

(21) Appl. No.: 10/793,389

(22) Filed: Mar. 3, 2004

Related U.S. Application Data
(60) Provisional application No. 60/451,525, filed on Mar. 3, 2003.

(51) Int. Cl. A01K 67/027; C07H 21/04; C12N 15/85

(52) U.S. Cl. 800/18; 435/455; 435/320.1; 536/23.2

(57) ABSTRACT

The invention provides non-human transgenic animals having conditional mdm2 transgenes integrated into their genome, cells derived therefrom, and methods for making and using both.

### CLAIM OF PRIORITY

This application claims the benefit under 35 USC §119(e) of U.S. Provisional Patent Application Serial No. 60/451,525, filed on Mar. 3, 2003, the entire contents of which are hereby incorporated by reference herein.

### FEDERALLY SPONSORED RESEARCH OR DEVELOPMENT

This invention was made with Government support under Grant No. 1R01CA077735-01A1 awarded by the National Institutes of Health. The Government has certain rights in the invention.

### TECHNICAL FIELD

This invention relates to the regulation of mdm2 function.

### BACKGROUND

The p53 tumor suppressor protein plays an essential role in regulating cellular growth and apoptosis, and is involved in the cellular response to stresses such as DNA damage and hypoxia. Activation of p53 can lead to cell cycle arrest or to apoptosis, and loss of p53 function is associated with tumorigenesis and genomic instability. Additionally, p53 is involved in the regulation of differentiation, senescence, and angiogenesis. p53 expression thus prevents the unchecked replication of cells with damaged DNA and guards against the accumulation of potentially oncogenic mutations. At least in part, p53 exerts its anti-tumor effects by acting as a transcription factor; a number of genes have been identified that are regulated at the transcriptional level by p53, including genes in the cell cycle arrest pathway and genes involved in the apoptotic response.

Rapid degradation through ubiquitin-dependent processes appears to be important in the maintenance of low levels of p53 protein. Degradation of p53 is regulated by binding of the proto-oncogene mdm2, which is amplified in approximately one-third of human sarcomas and overexpressed in a variety of other human tumors (reviewed in Woods and Vousden, Exp. Cell. Res. 264:56-66 (2001)). Overexpression of the mouse mdm2 cDNA or full-length gene induces tumorigenesis in transgenic mice (Lundgren et al., Genes Dev. 11:714-725 (1997); Jones et al., Proc. Natl. Acad. Sci. USA 96:15608-15612 (1998)).

### SUMMARY

The present invention is based, in part, on the discovery that conditional knockdown techniques can be used to create a transgenic animal in which expression of the mdm2 gene can be selectively suppressed in a temporally-controlled manner in the whole animal or in specific cells or tissues within the living animal or derived therefrom. The present invention provides transgenic animals, mdm2 gene targeting vectors, and cells comprising conditional mdm2 alleles.

In one aspect, the invention provides an mdm2 gene targeting vector. The vector includes some or all of the following: a first targeting sequence substantially identical to a DNA sequence 5′ of one or more exons of the mdm2 gene; a first recombinase recognition sequence; a second targeting sequence substantially identical to a DNA sequence of one or more exons of the mdm2 gene; a second recombinase recognition sequence; and a third targeting sequence substantially identical to a DNA sequence 3′ of one or more exons of the mdm2 gene. As used herein, “substantially identical” refers to a nucleotide sequence that contains a sufficient or minimum number of identical or equivalent nucleotides to the sequence of Mdm2, such that homologous recombination can occur. For example, nucleotide sequences that are at least about 75% identical to the sequence of Mdm2 are defined herein as substantially identical. In some embodiments, the nucleotide sequences are about 80%, 85%, 90%, 95%, 99% or 100% identical.

To determine the percent identity of two nucleic acid sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100% of the length of the reference sequence) is aligned. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

In some embodiments, the vectors described herein can include one or more selection markers, including, but not limited to, negative selection markers, e.g., the MC1-TK thymidine kinase negative selection marker, and/or antibiotic resistance genes, e.g., a neomycin resistance gene such as PGK-Neo.

In some embodiments, the targeting sequence substantially identical to a DNA sequence of one or more exons of the mdm2 gene can comprise the eleventh and/or twelfth exons of the mdm2 gene.

In certain embodiments, the recombinase recognition sequence is a lox sequence such as LoxP, Lox 66, Lox 71, Lox 511, Lox 512, and Lox 514, or a variant thereof. In some embodiments, the recombinase recognition sequence is an FRT sequence.

The invention also provides methods for producing transgenic animals, e.g., mammals, e.g., rodents, e.g., mice, whose somatic and germ cells comprise a conditional mdm2 allele. The method includes some or all of the following: transfecting an embryonic stem (ES) cell, e.g., a murine ES cell, in vitro with a targeting vector, e.g., as described herein; and generating a transgenic animal from the ES cell. Methods known in the art, and those described herein, can be used to transfect the ES cell and to generate the transgenic animal.

In addition, the invention provides one or more cells, e.g., isolated cells and cell lines, transfected with any one or more of the targeting vectors of the invention. In some embodiments, the cell is an ES cell.

The invention further provides a non-human transgenic animal, e.g., a mammal, e.g., a rodent, e.g., a mouse, whose somatic and germ cells comprise a conditional mdm2 allele. The conditional mdm2 allele can include some or all of the following: a first recombinase recognition sequence 5′ of at least one exon of an mdm2 gene, and a second recombinase recognition sequence 3′ of at least one exon of the mdm2 sequence. In some embodiments, the animal (and cells, tissue and organs derived therefrom) exhibits decreased mdm2 expression upon exposure to a recombinase, e.g., a Cre recombinase (e.g., from bacteriophage P1) or a functional variant thereof, or a Flp recombinase (e.g., from yeast) or a functional variant thereof. In some embodiments, some or all of the somatic and germ cells of the transgenic animal (e.g., the genome of the cells) include two conditional mdm2 alleles, each having a first recombinase recognition sequence 5′ of at least one exon of the mdm2 gene and a second recombinase recognition sequence 3′ of at least one exon of the mdm2 sequence, wherein the animal exhibits decreased mdm2 expression upon exposure to a recombinase, e.g., a Cre recombinase (e.g., from bacteriophage P1) or a functional variant thereof, or a Flp recombinase (e.g., from yeast) or a functional variant thereof.

In some embodiments, the exposure to Cre recombinase occurs as a result of administration of, for example, a recombinase expression vector (e.g., a virus) to the animal or cells, tissue and organs derived therefrom. In some embodiments, the exposure to the recombinase occurs as a result of expression of the recombinase, e.g., under the control of an inducible promoter (such as the interferon (IFN)-inducible MX1 promoter), a developmentally regulated promoter, or a tissue- or cell-specific promoter. In some embodiments, the exposure to the recombinase occurs as a result of the activation of the recombinase, e.g., by translocation of the recombinase to the nucleus.

In another aspect, the invention also includes cells, e.g., one or more isolated cells, tissues, or organs derived from the non-human transgenic animals of the invention. In some embodiments, the cell is a fibroblast, e.g., a mouse embryonic fibroblast (MEF).

The animals and cells of the present invention are useful as models for testing the effects of drugs that target p53 in the absence of Mdm2. Further, since Mdm2 is a potential target for cancer therapeutics, the animals and cells are useful as a model system in which Mdm2 is suppressed, both to investigate possible side effects of agents that suppress Mdm2 or cause dissociation of the Mdm2-p53 complex, and to provide a model of a system in which Mdm2 has been suppressed for use as a reference in designing drugs that target Mdm2. Additionally, removal of Mdm2 results in stabilized “activated” p53 which has been demonstrated to cause early aging phenotypes in vivo. Thus, the animals and cells of the invention are useful as a system for the development of anti-aging drugs, as well as agents effective in aiding wound healing, as in the case of the K5-Cre crosses. Furthermore, the effect of Mdm2-suppressing drugs on various embryonic stages of development can also be investigated, providing a system for examining the effects of p53-stabilizing drugs given to pregnant mothers.

The animals of the present invention have several advantages. First, in vivo Mdm2 suppression cannot be achieved using standard knockout techniques, as mdm2 null mice do not develop; the mdm2 knockout causes embryonic lethality. The conditional nature of the present invention allows for the evaluation of Mdm2 suppression in the adult animal. The conditional expression system allows for temporally or spatially targeted suppression of Mdm2. Additionally, the conditional nature of the knockout provides for more relevant controls, in which the recombinase is absent. The role of Mdm2, and the effect of suppression of Mdm2, in various embryonic stages of development can be investigated by crossing the mdm2^{cndl/cndl} mice described herein with other transgenic mice. For example, the developmental consequence of reduced Mdm2/elevated p53 can be determined in vivo by crossing Mdm2 conditional mice, e.g., as described herein, with transgenic mice expressing a recombinase under the control of a promoter that turns on at a certain time, e.g., after the determined embryonic lethality time point at embryonic day 5.5 (day e5.5). The effect of suppression of Mdm2 in the adult animal can also be evaluated by administration of a recombinase, e.g., purified recombinase protein, or by administration of a nucleic acid expressing the recombinase, e.g., a virus. This provides a system for examining the effects of p53-stabilizing drugs given to pregnant mothers. This system can be used for studying wound healing as well, as p53 has a major role in repairing damaged tissues.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic drawing of one embodiment of an mdm2 gene targeting vector (middle row) and the mdm2 gene before (top row) and after (bottom row) homologous recombination. The gene replacement vector contained 2.9 Kbp of 5′ sequence homology, 2.4 Kbp of 3′ sequence homology, two loxP sites (filled triangles), a PGK-NEO cassette (“NEO”), and an MC1-TK negative selection marker (“TK”). Expected recombination sites are marked “R”. Exons number 9-12 are shown as squares or rectangles with the appropriate number.

FIG. 2 is an autoradiograph of a southern blot of digested genomic DNA from mice heterozygous for the conditional mdm2 and the wild-type (wt) allele (mdm2^{condl/+}); homozygous for the conditional mdm2 (mdm2^{condl/condl}); or heterozygous for the conditional mdm2 and a null allele (mdm2^{condl/ml})

FIG. 3 is an autoradiograph of a Northern blot of total RNA isolated from mdm2^{condl/ml-} embryonic fibroblasts at the time shown and probed for mdm2 or Gapdh (control).

FIG. 4 is a photograph of mdm2 or Gapdh (control) RT-PCR products separated electrophoretically. Cre, Cre recombinase (lanes in which Cre recombinase was present are indicated by a “+”); RT, reverse transcriptase (lanes in which RT was present are indicated by a “+”); M, marker. The lanes are numbered below for reference.

FIGS. 5A-5D are a series of photomicrographs of MEF cells 96 hours after transduction with 60,000 infectious particles of Ad-βgalactosidase virus (5A and 5B) or Ad-Cre virus (5C and 5D). FIGS. 5A and 5C are control wildtype MEF cells, and FIGS. 5B and 5D are mdm2^{cndl/cndl} MEF cells.

FIGS. 6A-6D are a series of photomicrographs of cells 96 hours after transduction with 60,000 infectious particles of Ad-β-galactosidase virus (6A, 6C, and 6E) or Ad-Cre virus (6B, 6D, and 6F). FIGS. 6A and 6B are control wildtype MEF cells, and FIGS. 6E and 6F are mdm2^{cndl/ml} adult somatic ear fibroblasts, 6C and 6D, and mdm2^{cndl/ml} mouse embryonic fibroblasts.

FIG. 7A is a photomicrograph of mdm2^{cndl/cndl}-p21−/− MEFs 48 hours after transduction with 60,000 infectious particles of Ad-βgalactosidase virus.

FIG. 7B is a photomicrograph of mdm2^{cndl/cndl}-p21−/− MEFs 48 hours after transduction with 60,000 infectious particles of Ad-Cre virus.

FIGS. 8A-8D are a series of photomicrographs of crystal violet stained 10 cm plates of MEFs 96 hours after transduction with 60,000 infectious particles of Ad-Bgal (8A and 8C) or Ad-Cre (8B and 8D). FIGS. 8A and 8B are Mdm2^{cndl/cndl}-p21^{-/-} MEFS, and FIGS. 8C and 8D are Mdm2^{cndl/cndl} MEFs.

FIG. 9A is a cell cycle profile of Mdm2^{cndl/cndl} MEFs at the time of viral infection following FACS analysis.

FIG. 9B is a cell cycle profile of Mdm2^{cndl/cndl} MEFs 72 hours following viral infection with Ad-Cre using FACS analysis. The subG0 fraction represents the number of apoptotic cells when stained with Propidium Iodide.

FIG. 10 is a bar graph representing the percent of cells in G1 at 48 hours after infection with the Ad-Cre virus. Cell type is indicated below each bar.

### DETAILED DESCRIPTION

The present invention is based, in part, on the discovery that the mdm2 gene can be altered to be conditionally knocked out via Cre-mediated site-specific recombination of a cassette encompassing a portion of the mdm2 gene flanked by two lox sites (also referred to as “floxed”). The lox (locus of X-ing over) sites are sequences, derived from phage DNA, that are recognized by the Cre recombinase (causes recombination) (Sternberg et al., J. Mol. Biol. 150:467-486 (1981)). In the absence of Cre recombinase, the mdm2 gene is expressed and functions normally. In the presence of Cre recombinase, recombination of the lox-mdm2 cassette leads to loss of mdm2 transcripts.

The oncogenic potential of Mdm2 is believed to be due, in part, to the ability of the Mdm2 protein to bind to the p53 tumor suppressor protein. Mdm2-p53 complex formation has been found to inhibit p53-mediated transactivation of gene expression; Mdm2 acts as an E3-ubiquitin ligase to target p53 for degradation in the proteosome (reviewed in Zhang and Xiong, Cell Growth Differ. 12:175-186 (1999)). In mice, mdm2 deficiency leads to early embryonic lethality during gestation due to p53-induced cell growth arrest or apoptosis, whereas mice deleted for both mdm2 and p53 are viable (Jones et al., Nature 378:206-208 (1995); Montes de Oca Luna et al., Nature 378 203-206 (1995)). Mdm2-p53 double null mouse embryonic fibroblasts have been useful for studying the functional interactions of Mdm2 and p53. However, a limitation to these studies is the absence of endogenous p53 (Jones et al., Proc. Natl. Acad. Sci. USA 93:14106-14111 (1996); McMasters et al., Oncogene 13:1731-1736 (1996)). An understanding of the Mdm2-dependent and -independent roles of p53 in regulating cell growth and development would allow for the development and evaluation of, e.g., therapeutic and diagnostic tools. Thus, a conditional mdm2 mouse allele was generated using gene-targeting technology in mouse embryonic stem (ES) cells.

Transgenic Animals and Methods of Use

The invention provides non-human transgenic animals having conditional mdm2 transgenes integrated into their genome. In some embodiments, the conditional transgenes make use of the Cre-lox system or the Flp-FRT system.

A “transgenic animal” is a non-human animal, such as a mammal, generally a rodent such as a rat or mouse, in which one or more of the cells of the animal includes a transgene. Other examples of transgenic animals include non-human primates, sheep, dogs, cows, goats, chickens, amphibians, and the like. A “transgene” is exogenous DNA that is integrated into the genome of a cell from which a transgenic animal develops and which remains in the genome of the mature animal, thereby directing the expression of an encoded gene product in one or more cell types or tissues of the transgenic animal. As used herein, an “homologously recombinant animal” is a non-human transgenic animal, such as a mammal, typically a mouse, in which an endogenous mdm2 gene has been altered by homologous recombination between the endogenous gene and an exogenous DNA molecule introduced into a cell of the animal, e.g., an embryonic cell of the animal, prior to completed development of the animal.

A “conditional transgene” as used herein refers to an insertion of recombinase recognition sequences into endogenous chromosomal DNA, e.g., an mdm2 gene, which is integrated into or occurs in the genome of the cells of a transgenic animal. A “recombinase recognition sequence” as used herein refers to a sequence that directs the recombinase-mediated excision or rearrangement of DNA. Such sequences include lox and FRT sequences, as described herein. The conditional transgene can suppress the expression of the mdm2 gene product in one or more cell types or tissues of the transgenic animal, e.g., knockout or reduce expression, in the presence of the Cre recombinase. “Suppression of gene expression” includes both complete suppression and partial suppression, sunpression under specific circumstances (e.g., temporally or spatially limited suppression), and suppression of one or both alleles of a gene. Expression can be monitored by any method known in the art, and can be measured by assaying RNA, protein, or activity. Thus, a transgenic animal can be one in which an endogenous mdm2 gene has been altered, e.g., by homologous recombination between the endogenous gene and an exogenous DNA molecule (such as the mdm2 gene targeting vectors of the invention) introduced into a cell of the animal, e.g., an embryonic cell of the animal, prior to development of the animal. A line of transgenic animals (e.g., mice, rats, guinea pigs, hamsters, rabbits, or other mammals) can be produced bearing a transgene encoding a conditional mdm2. Methods known in the art for generating such transgenic animals would be used, e.g., as described below.

The use of the Cre-lox system to direct site-specific recombination in transgenic animals is described in Orban et al., Proc. Natl. Acad. Sci. USA 89(15):6861-5 (1992); Akagi et al., Nuc. Acids Res. 25(9):1766-1772 (1997); Lakso et al., Proc. Natl. Acad. Sci. USA 89:6232-6236 (1992); Rossant and McMahon, Genes Dev. 13(2)142-145 (1999); Wang et al., Proc. Natl. Acad. Sci. USA 93:3932-3936 (1996). The use of the Flp-FRT system to direct site-specific recombination in transgenic animals is described in U.S. Ser. No. 08/866,279, Publication No. U.S. 2002/0170076; Vooijs et al., Oncogene. 17(1):1-12 (1998); Ludwig et al., Transgenic Res. 5(6):385-95 (1996); and Dymecki et al., Dev. Biol. 201(1):57-65 (1998). Methods for transgenic animals can be used to generate an animal, e.g., a mouse, that bears one conditional mdm2 allele and one wild type mdm2 allele. Two such heterozygous animals can be crossed to produce offspring that are homozygous for the conditional allele.

For example, in one embodiment, recombinase recognition sequences are introduced into an endogenous mdm2 gene of a cell, e.g., a fertilized oocyte or an embryonic stem cell. Such cells can then be used to create non-human transgenic animals in which conditionally regulated mdm2 sequences have been introduced into their genome, e.g., homologously recombinant animals in which endogenous mdm2 nucleic acid sequences have been rendered conditional. Such animals are useful for studying the function and/or activity of mdm2 and for identifying and/or evaluating modulators of mdm2 and/or p53 function, as well as the functional consequences of downregulating or eliminating mdm2 activity in an adult animal.

Methods for generating transgenic animals, particularly animals such as mice, via embryo manipulation and electroporation or microinjection of pluripotent stem cells or oocytes, are known in the art and are described, for example, in U.S. Pat. Nos. 4,736,866 and 4,870,009, U.S. Pat. No. 4,873,191, U.S. Ser. No. 10/006,611, and in Hogan, Manipulating the Mouse Embryo, (Cold Spring Harbor Laboratory Press, Cold Spring Harbor, N.Y., 1986), which are incorporated herein by reference in their entirety. Retroviral vectors can also be used, as described in Robertson et al., Nature 323:445-448 (1986). Retroviruses generally integrate into the host genome with no rearrangements of flanking sequences, which is not always the case when DNA is introduced by microinjection or other methods. Methods similar to those used to create transgenic mice can be used for production of other transgenic animals. A transgenic founder animal can be identified based upon the presence of the conditional Mdm2 transgene in its genome, for example by detecting the presence of the recombinase recognition sequences (e.g., lox or FRT). Founder animals can also be identified by detecting the presence or expression of the conditional Mdm2 mRNA in tissues or cells of the animals in the presence and/or absence of recombinase, e.g., Cre or Flp. For example, fibroblasts can be used, such as embryonic fibroblasts or fibroblasts derived from the post-natal animal, e.g., the ear of the post-natal animal. A transgenic founder animal can then be used to breed additional animals carrying the transgene. Moreover, transgenic animals carrying a transgene encoding a conditional mdm2 gene can further be bred to other transgenic animals carrying other transgenes.

An advantage of generating conditional Mdm2 animals is that in the absence of conditional induction, mice are free of deleterious effects that can interfere with their propagation.

The Mdm2 conditional transgenic animals described herein are useful for studying the function and/or activity of mdm2, as well as for evaluating the effects of downregulating or eliminating mdm2, in the presence and/or absence of other genes and agents. For example, if Mdm2 is selectively removed from the epidermis using the K5 Cre system, the effects of p53 overexpression on wound healing, aging, etc. can be examined, and compounds for use in treating wounds or aging-related disorders can be evaluated. Once a phenotype has been elicited through the removal of Mdm2, the efficacy of various drugs for reversing the assumed phenotype can be assessed. For example, the present cells and animals provide powerful tools for assessing the physiological consequences of deregulated p53, e.g., p53 deregulated by drugs. The use of drugs that interfere with the binding of Mdm2 to p53 will presumably lead to elevated levels of p53, which may ultimately result in premature aging for the patient. Any drug intended to target the Mdm2/p53 interaction can be screened for potential unintended effects. The animals described herein can thus be used to identify and/or evaluate side effects of negative modulators of mdm2 activity.

In addition, the Mdm2 conditional animals described herein can be used to identify or evaluate modulators of p53 or other genes in the presence and absence of mdm2, and to evaluate the effects of suppression of Mdm2 expression or function, e.g., in adult animals or in temporally- and spatially-defined cells, tissues, or organs, in combination with the presence or absence of other genes or agents. For example, the Mdm2 conditional mice or cells (e.g., cells that have been exposed to recombinase, e.g., cells in which the expression of a recombinase has been induced, including cells containing either the K5-Cre, MX1-Cre, or other recombinase-expressing transgenes or nucleic acids, or cells to which a recombinase protein has been added) can be exposed to a stimulus, e.g., administration of a DNA-damaging stimulus such as ionizing, gamma, or UV irradiation, or a transforming stimulus, e.g., infection with an activated ras plus myc transforming retrovirus, to elicit a response, e.g., a p53-mediated response, e.g., apoptosis or transformation. Responses can be compared, for example, between mice (or tissues, organs, or cells derived therefrom) that contain (e.g., express) functional Mdm2 and those that lack functional Mdm2, in the presence and absence of one or more test compounds, to evaluate the ability of the test compounds to increase or diminish a response, e.g., a p53-mediated response, further stabilize p53, and/or specifically interfere with a mutant variant of p53, e.g., the mutant p53 in older mice that have been continually exposed to DNA damaging reagents. In some embodiments, the stimulus is the deletion or reduction of expression of a gene, e.g., p53 or p21. In some embodiments, the response is associated with cellular transformation, e.g., proliferation, tumor formation and growth, and metastasis. In some embodiments, the response is associated with aging, e.g., hair sparseness, reduced dermal thickness, reductions in bone density, lordokyphosis, lymphoid atrophy, body mass, lifespan, organ mass, wound healing, and subcutaneous adipose. A number of suitable assays are known in the art for evaluating aging-associated responses and wound healing, see, for example, Tyner et al., Nature 415(6867):45-53 (2002).

Mdm2 Gene Targeting Vectors

Also provided herein are gene targeting DNA constructs. In some embodiments, to create a homologously recombinant animal, a vector is prepared that contains a portion of an mdm2 gene, into which first and second recombinase recognition sequences, e.g., lox or FRT sequences, are inserted, flanking a region of the mdm2 gene, e.g., the eleventh (SEQ ID NO:4) and twelfth exons (SEQ ID NO:6). In general, the recombinase recognition sequences are inserted into an intron to minimize any disruption of the gene; if they are placed in an intron, they should be in frame with the gene sequence and not cause disruptions downstream. The two recombinase recognition sequences can be in the same orientation within the endogenous gene locus, to cause a deletion, or in the opposite orientation, to generate a null allele via inversion. Both recombinase recognition sequences should be for the same recombinase, e.g., both lox or both FRT. The lox sequence can be any lox sequence known in the art, including, but not limited to, loxP, lox66, lox71, lox51, lox512, and lox514, as well as variants and mutants thereof, such as those described in U.S. Pat. No. 6,465,254. For example, the lox sequence can be loxP (gcttgggctgcaggtcgagggacctaataacttcgtatagcatacattatacgaagttatattaagggttccggatcccgg) (SEQ ID NO:8). The FRT sequence can be any FRT sequence known in the art (see, e.g., Fiering et al., Proc. Natl. Acad. Sci. U.S.A. 90(18):8469-73 (1993)).

Typically, the vector is designed such that, upon homologous recombination, the endogenous mdm2 gene can be conditionally knocked out, e.g., is knocked out under conditions in which a recombinase (such as Cre or Flp) is present. Typically, in the homologous recombination vector, a portion of the mdm2 gene sequence or regulatory region thereof is flanked at its 5′ and 3′ ends by lox sequences, which in turn are flanked by additional nucleic acid of the mdm2 gene to allow for homologous recombination to occur between the exogenous mdm2 conditional nucleic acid sequence carried by the vector and an endogenous mdm2 gene in an embryonic stem cell. This creates a targeting vector with a structure as follows:

(a) a first targeting sequence that is substantially identical to a DNA sequence 5′ of one or more exons of the mdm2 gene, flanking the floxed region on the 5′ end;

(b) a first recombinase recognition sequence, e.g., a lox or FLT sequence;

(c) a second targeting sequence substantially identical to a DNA sequence of one or more exons of the mdm2 gene, which forms the floxed region that is excised or inverted by the recombinase;

(d) a second recombinase recognition sequence e.g., a lox or FLT sequence to match the first recombinase recognition sequences (e.g., both are lox or both are FLT); and

(e) a third targeting sequence substantially identical to a DNA sequence 3′ of one or more exons of the mdm2 gene, flanking the floxed region on the 3′ end.

The targeting sequences substantially identical to the sequence of mdm2 nucleic acid that flank the floxed region are of sufficient length for successful homologous recombination with the endogenous gene. In some embodiments, at least 400 bp of flanking DNA that is substantially identical to sequences 5′ and/or 3′ of the floxed region will be included in the vector (at both the 5′ and 3′ ends or “arms”); typically, several kilobases of flanking DNA will be included in each arm of the vector (see, e.g., Thomas and Capecchi, Cell 51:503-12 (1987) for a description of homologous recombination vectors).

In general, the combined length of the substantially identical regions of both 5′ and 3′ arms will be greater than about 2 Kb, e.g., about 3-8 Kb or more. The substantially identical regions can be unequally distributed, e.g., 0.5 Kb homology on the 5′ arm and 1.5 Kb homology on the 3′ arm, or equally distributed. Greater degrees of homology will generally result in more successful recombination, however, some variation is tolerated, e.g., PCR generated arms can be used which may incorporate missense mutations. For example, the vector can contain regions of homology from intron 9 (SEQ ID NO:1), exon 10 (SEQ ID NO:2); intron 10 (SEQ ID NO:3); exon 11(SEQ ID NO:4); intron 11 (SEQ ID NO:5); exon 12 (SEQ ID NO:6); and/or the polyA sequence and region downstream of exon 12 (SEQ ID NO:7). The vector can also contain positive and negative selection markers, including, but not limited to, drug resistance genes such as a neomycin resistance gene or thymidine kinase gene; toxins such as the diphtheria toxin (DT) A gene; or any combination thereof. Such markers are useful for selection of those cells that have the desired homologous recombination product, such as selecting ES cells before implantation, as described below in Example 2.

The vector can be introduced into an embryonic stem cell line (e.g., by electroporation), and cells in which the introduced conditional mdm2 sequence has homologously recombined with the endogenous mdm2 gene are selected, e.g., by antibiotic selection (see, e.g., Li et al., Cell 69:915-926 (1992)). Selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, Current Opinion in Bio/Technology 2:823-829 (1991) and in PCT Publication Nos. WO 90/11354, WO 91/01140, WO 92/0968, and WO 93/04169. One method of producing such mice is described in Example 2 herein.

Suppression of Mdm2 can be achieved in the conditional transgenic animals described herein (and tissues, organs and cells derived therefrom) by exposure to an appropriate recombinase (e.g., Cre or Flp). This exposure can be achieved by expression of the recombinase (e.g., Cre or Flp) in a cell that contains the conditional transgene. For example, a vector (e.g., a viral vector, see, e.g., Psarras et al., J. Gene Med. 6(1):32-42 (2004); Thevenot et al., (Mol. Cell. Neurosci. 24(1):139-147 (2003); and Nakano et al., Nuc. Acids Res. 29: e40, 2001) for expressing the recombinase can be introduced into the cell. Any suitable delivery method can be used; in some embodiments, delivery can be targeted to specific cells or tissues, including oral administration, nasal administration, or parenteral administration, including tail vein injection. See, e.g., Jackson et al., Genes and Dev. 15:3243-3248 (2001); Chan et al., J. Clin. Inv. 113(4):528-538 (2004); Wood et al., Cancer Gene Therapy 6(4)367-372 (1999). In some embodiments, Cre recombinase can be delivered directly into the cells, e.g., as a purified protein. In some embodiments, the Cre recombinase is delivered as TAT-NLS-Cre, a fusion protein with TAT peptide (an Arg rich peptide derived from HIV) and nuclear localizing signal (NLS) (Kasim et al., Nuc. Acids Res. Suppl. 3:255-256 (2003)). In some embodiments, the Mdm2 conditional mice described herein are crossed with mice expressing a recombinase, e.g., under a promoter that controls the expression or activity of the recombinase, e.g., spatially or temporally. For example, the mice expressing the recombinase can be MX1Cre mice. The MX1Cre mice express Cre recombinase under the control of the IFN-inducible MX1 promoter (Kuhn et al., Science 269(5229):1427-9 (1995); Schneider et al., Am. J. Renal Physiol. 284:F411-F417 (2003)). A increase in IFN levels induces the expression of Cre in the cells of these animals. In some embodiments, recombinase expression from the MX1 promoter is induced in vivo by treating mice (or tissues, organs, or cells derived therefrom) with IFN-α/β or pI-pC, a synthetic double-stranded RNA that induces expression of endogenous IFN (Chan et al., supra). In some embodiments, a fusion protein consisting of recombinase and a truncated progesterone receptor is expressed (see, e.g., Zhou et al., Genesis 32:191-192 (2002)); the presence of the progesterone receptor fragment targets the fusion protein to the epidermis. The fusion protein remains sequestered in the cytoplasm, where the recombinase is inactive, until a progesterone antagonist, such as RU486, induces translocation into the nucleus, where the recombinase excises DNA sequences that have been flanked by recombinase recognition sites.

The cells of the invention can be isolated from a transgenic, animal using methods known in the art, or can be created by transfecting or transforming cells, such as cultured cells, e.g., fibroblasts, thymocytes, neurons, glia, or ES cells, inter alia, with the mdm2 gene targeting vectors described herein using known methods. For example, vector DNA can be introduced into host cells via conventional transformation, transduction, or transfection techniques. As used herein, the terms “transduction,” “transformation,” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection, and infection.

Conditionally removing Mdm2 in both somatic adult fibroblasts and mouse embryonic fibroblasts results in a unique system to examine the developmental and genetic effects of the loss of Mdm2 function, for example, on the requirements for eliciting apoptosis. In MEFs, RNA can be isolated at various time points following infection to determine which gene messages are up- or down-regulated during the apoptotic response, thereby identifying further potential drug targets for eliciting or forestalling apoptosis. Various transgenes or compounds can be introduced into this system to assess their effects on apoptosis. Additionally, downstream genes of p53 that are required for apoptosis to occur can be identified by comparing genes activated or downregulated in somatic versus embryonic fibroblasts, using methods known in the art, such as subtractive methods including gene chips and arrays, inter alia. Thymocytes can also be used, e.g., for examining apoptosis in response to gamma irradiation, UV, or other forms of DNA damage. Suitable thymocytes can be, for example, thymocytes harvested between 4-8 weeks post birth. Tumor cell lines may be derived from animals exhibiting tumor formation. Reactive gliosis can also be examined in astrocyte cultures, for example, astrocytes and neurons can be removed, e.g., from p0 (newborn) mice, and studied in vitro.

### EXAMPLES

The invention is further described in the following examples, which do not limit the scope of the invention described in the claims.

### Example 1

### Construction of an mdm2 Conditional Allele

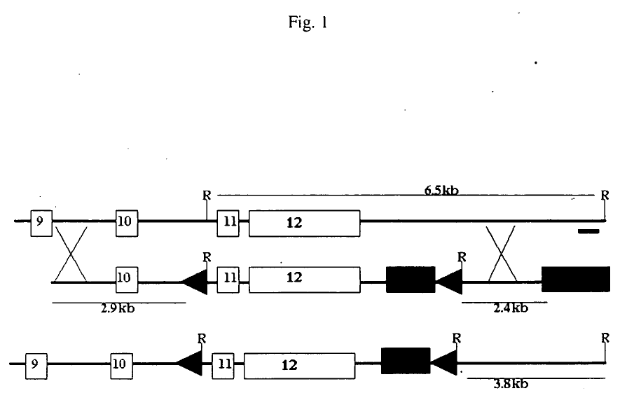

The Cre/LoxP recombination system (see, e.g., Orban et al., supra) was used to design an mdm2 conditional allele (mdm2^{cndl}). As shown in FIG. 1, the gene replacement vector contained 2.9 Kbp of 5′ sequence substantially identical to the mdm2 gene, 2.4 Kbp of 3′ sequence substantially identical to the mdm2 gene, two loxP sites (filled triangles), a PGK-NEO cassette (“NEO”), and an MC1-TK negative selection marker (“TK”). PGK-NEO is a hybrid gene in which the phosphoglycerate kinase I promoter drives the neomycin phosphotransferase gene. The first loxP site was inserted 5! to exons 11 and 12 of the mdm2 gene and a neomycin reporter gene cassette, and a second loxP motif was inserted 3′ of the neomycin reporter gene and exon 12, the final exon of the mdm2 gene. The vector was used in gene-targeting experiments in AB2.2 ES cells. Proper targeting of the Mdm2 locus (to chromosome 10, c1) placed the neomycin marker cassette 560 bp downstream of the mdm2 polyadenylation signal and in a reverse orientation with respect to the mdm2 gene, thus the presence of the cassette had no effect on Mdm2 expression (Jones et al., Gene 175:209-213 (1996); Mendrysa et al., Gene 264:139-146 (2001)).

### Example 2

### Generation of mdm2^{cndl} Hetero- and Homozygous Mice

The mdm2^{cndl} targeting construct was introduced into AB2.2 ES cells (129/SvEvBrd) by electroporation. Following drug selection for 10 days in 180 ug/ml Geneticin (G418) and 0.2 μm FIAU supplemented 15% FBS/DMEM/GPS, cells were grown as clones. 1×10⁷ cells were trypsinized, electroporated with 10 μgs of linearized construct DNA, and plated onto 2-100 cm tissue culture dishes containing mitotically inactive “feeder cells,” fibroblast cells that have been growth arrested with Mitomycin C Treatment, which provide necessary secreted factors that inhibit differentiation of the ES cells.

Following both positive (G418) and negative (FIAU) drug selection for 10-14 days, the remaining suriving clones were isolated and screened for correct integration of the construct. Four clones that had properly incorporated the construct were identified via Southern blot analysis using 5′ and 3′ external probes that hybridize to regions outside of the original targeting construct. The 5′ probe was a 700 bp BamHI-Kpn1 digested fragment of genomic DNA containing intron 6 of the Mdm2 gene-used on a Southern with Spe1 digested genomic DNA which yields a wild type band of 13 kb and a targeted band of 10 kb. The 3′ end was screened using an external probe generated by PCR using EcoR1 digested DNA as a template, with primers EXTF 5′ GCTTACTATGAGTTCCAAGTGCAG 3′ (SEQ ID NO:9) and EXTR 5′ CCTGGACTAGCCATCTTAGAA 3′ (SEQ ID NO:10). The wild type appeared as a band of about 6.5 Kb and the targeted allele was about 3.9 Kb. The use of an external probe prevented the false positive detection of random integrants. Proper targeting of the Mdm2 gene was confirmed by Southern analysis of EcoR1-digested genomic DNA using a 3′ external probe (rectangle on FIG. 1), and further confirmed by Southern analysis of Spe1-digested genomic DNA using a 5′ external probe.

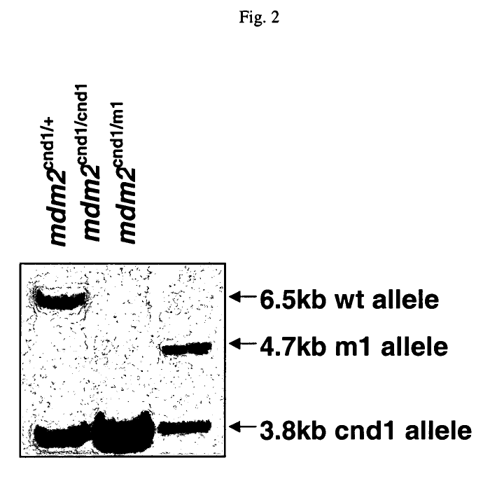

Successfully targeted mdm2 genes are termed “floxed.” Clone A1G was used in blastocyst injection experiments to generate six high-degree chimeric mice, e.g., mice having a high percentage of “successful” coat color. The ES cells were from mice having the dominant agouti coat color genes, whereas the adoptive blastocysts will generate black mice if left unmanipulated. High degree chimeras had greater than 80% agouti coat color which suggested penetrance of the transgene to germline tissues. These mice were then used to backcross and the resulting offspring that were 100% agouti were considered to be representatives of “successful germline transmission.” Breeding of these chimeric male mice to C57B1/6 female mice yielded germ-line transmission of the mdm2^{cndl} allele into the F1 generation (FIG. 2).

To ensure that the generation of the conditional mdm2 allele did not disrupt normal mdm2 gene function, mdm2^{cndl} heterozygous mice were bred to generate homozygous offspring and also were crossed with heterozygous mdm2 non-conditional knockout (mdm2^{ml}) mice generated previously (Jones et al. (1995) Nature 378:206-208).

These matings gave rise to viable mdm2^{cndl/cndl} mice and to mdm2^{cndl/ml} mice (FIG. 2). Since homozygous mdm2-null mice are embryonic lethal, these results indicate that modification of mdm2 to generate the conditional allele did not disrupt mdm2 function. Furthermore, no abnormalities were observed in either mdm₂^{cndl/cndl} mice or mdm2^{cndl/ml} up to one year of age. Features that can be monitored to asses normal function include muscular strength, clasp reflex, coat maintenance, spontaneous tumor formation, early morbidity, and genetic transmission from generation to generation that does not conform to Mendalian rules of inheritance. Thus, the conditional mdm2 mouse is a useful model for the study of p53 regulation in the presence and absence of Mdm2 expression.

### Example 3

### Isolation of Cells

Embryonic Fibroblasts

Embryonic fibroblasts were generated from day e11.5-e14.5, mouse embryos including day e13.5 mdm2^{cndl/ml} and day e 13.5 mdm2^{cndl/cndl} mouse embryos. Briefly, pregnant females were sacrificed at day 13.5 of gestation. Embryos were removed and washed in phosphate buffered saline (PBS). After removal of small piece of tissue for genotyping, the embryos were placed into the barrel of a 1 ml syringe with an 18½ g needle and forcefully dispensed into a 15 ml conical tube. One ml of trypsin (2.5 mg/ml) was used to dissociate the tissue. Samples were placed into a 37° C. incubator, 5% CO₂ for 15 minutes. Cells were then placed into 100 mm tissue culture plates containing 10 ml Dulbecco's Modified Eagle's Medium (DMEM) supplemented with 15% fetal calf serum, penicillin (48.30 U/ml), and streptomycin (37.8 μg/ml), and L-glutamine (2 mM). These were considered passage 0 mouse embryonic fibroblasts (MEFs) and could be kept alive in culture up to about passage 6.

Adult Somatic Fibroblasts

Adult somatic ear fibroblasts were generated from severed ears from adult mice. Ears were placed in PBS, rinsed two times, and minced into pieces using a sharp sterile razor blade in a 100 mm tissue culture dish. DMEM supplemented with 10% fetal bovine serum, 2 mM L-glutamine, 48.3 U/ml penicillin and 37.8 μg/ml streptomycin was added to the ear cultures. The resulting fibroblasts typically required 4-6 days growth in culture in order to fill an entire 100 mm plate (2 ears/plate); normally, these cells survived in culture for up to about six passages.

### Example 4

### Confirmation of the Conditional Allele

To confirm the conditional nature of the floxed mdm2 allele prior to mating with Cre-expressing transgenic mice, mdm2^{cndl/ml} embryonic fibroblasts were generated from day e13.5 mdm2^{cndl/ml} mouse embryos. The fibroblasts were infected with a recombinant Cre-adenovirus to transduce Cre expression in these cells (Akagi et al., Nucleic Acids Res. 25:1766-1773 (1997)), and total RNA was isolated at various time points.

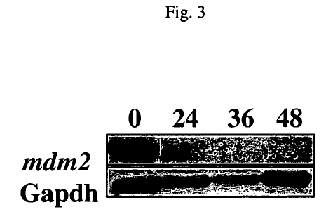

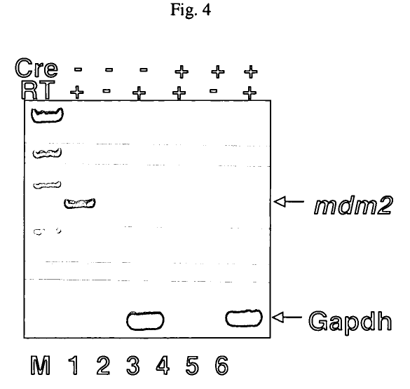

The presence of mdm2 mRNA was analyzed by Northern analysis of total RNA isolated from the mdm2^{cndl/ml} embryonic fibroblasts at 24, 36, or 48 hours following infection with Cre-adenovirus. mdm2 message was not detectable at 24 hours post-infection, as determined by both Northern analysis (FIG. 3) and by RT-PCR (FIG. 2) using a forward primer to mdm2 transcript sequences encoded in exon 3 (5′-ATGTGCAATACCAACATGTCTGTGTC-3′; SEQ ID NO:11) and a reverse primer to mdm2 sequences encoded in exon 12 (5′-GCAGATCACACATGGTTCGATGGCA-3′; SEQ ID NO:12) (FIG. 4), whereas control infection of mdm2^{cndl/ml} cells with a recombinant adenovirus encoding a β-galactosidase reporter gene had no effect on mdm2 MRNA levels.

RT-PCR using mdm2-specific or GAPDH-specific primers was performed on total RNA isolated from the cells at 24 hours post-infection with either a control adenovirus carrying the β-galactosidase gene (Ad-Gal, FIG. 4, lanes 1-3) or an adenovirus carrying the Cre recombinase gene (Ad-Cre, FIG. 4, lanes 4-6). In FIG. 4, RT(−) lanes are control reactions in which the RT-PCR was performed in the absence of reverse transcriptase. A 40-cycle PCR product of expected size was obtained from control infected cells but not from cells infected with the Ad-Cre virus. Without wishing to be bound by theory, it is possible that this elimination of mdm2 mRNA is due to destabilization of mdm2 mRNA upon deletion of the polyadenylation signals encoded in the 3′ UTR of Mdm2.

These results demonstrate (1) the creation of a conditional mdm2 allele that behaves as a null allele upon Cre-mediated excision of the floxed mdm2 exons, and (2) Cre-transduction induces loss of mdm2 transcripts in mdm2-conditional fibroblasts. Thus, these mdm2-conditional alleles are useful for analyzing the role of mdm2-mediated regulation of p53 in development and cell growth.

### Example 5

### Conditional Suppression of mdm2 Expression in mdm2^{cndl/cndl} MEFs

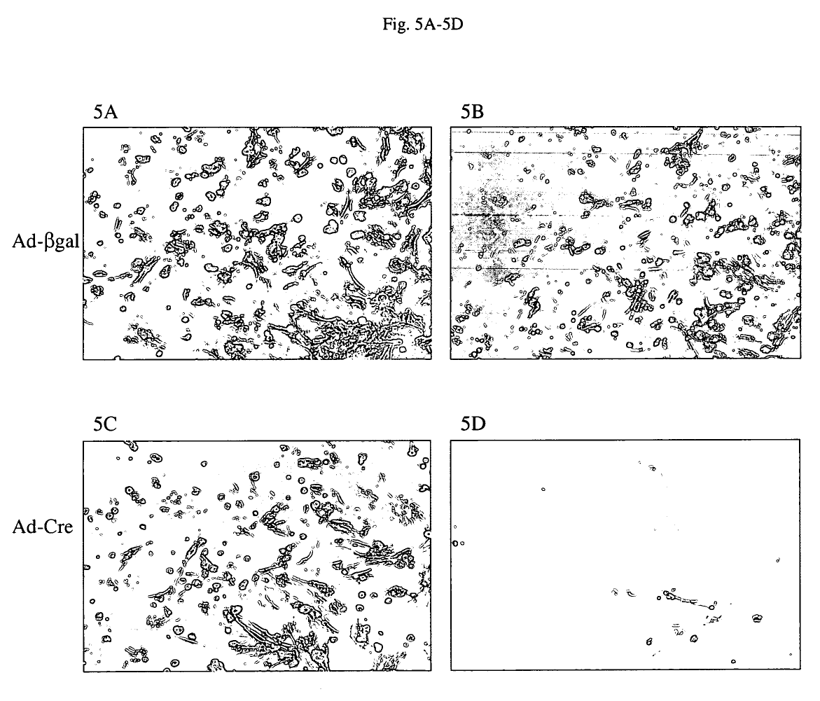

To evaluate the effect of suppression of mdm2 expression, control MEF cells and mdm2^{cndl/cndl} MEF cells were infected with Ad-Gal or Ad-Cre and cultured for 96 hours. As is shown in FIGS. 5A-5C, the control MEF cells infected with either virus were viable and showed no obvious phenotypic alterations. mdm2^{cndl/cndl} MEF cells infected with Ad-Gal were also viable and without obvious alterations (FIG. 5B). In contrast, the vast majority of mdm2^{cndl/cndl} MEF cells infected with Ad-Cre underwent apoptosis (FIG. 5D).

These data demonstrate that the loss of Mdm2 under physiological conditions (e.g., no overexpression in vitro studies or external cellular insults needed to be added to the system) results in apoptosis; as one theory, not meant to be limiting, this apoptosis is mediated by deregulated p53. Furthermore, the data illustrate a system for testing compounds that may facilitate or interfere with the p53-mediated apoptotic process, e.g., to test for drugs that down-regulate p53 to attempt to rescue the apoptosis phenotype. This system is also useful in identifying genes activated during the apoptotic process, which are potential targets for drugs that modulate the process.

### Example 6

### Conditional Suppression of mdm2 Expression in p21^{-/-} MEFs

The cyclin-dependent kinase inhibitor p21^{(Wafl/Cip1/Sdi1)}(“p21”) mediates the ability of p53 to arrest cellular proliferation, and cells derived from p21-null mice inefficiently arrest proliferation after p53 activation (Martin-Caballero et al., Cancer Res. 61:6234-6238 (2001). To evaluate the effect of suppressing expression of Mdm2 in MEF cells lacking p21, mdm2^{cndl/cndl}-p21^{-/-} mice were generated by crossing mdm2^{cndl} homozygous mice with p21⁻ homozygous mice obtained from Jackson Labs, stock number 003263, produced originally by Tyler Jacks (Nature (1995), 377:552-557. Briefly, mdm2^{cndl/cndl} mice were bred to p21 null mice which resulted in the production of mdm2^{cndl/+}-p21^{+/-} mice. The matings produced viable offspring. These mice were further backcrossed to p21 null mice resulting in mdm2^{cndl/+}-p21 null mice. Mdm2^{cndl/+}-p21 null mice were interbred to produce mdm2^{cndl/cndl}-p21^{-/-} mice, and embryonic fibroblasts were harvested from pregnant females.

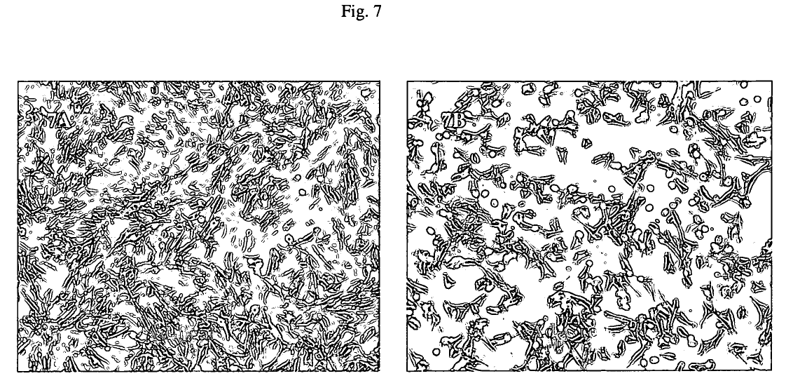

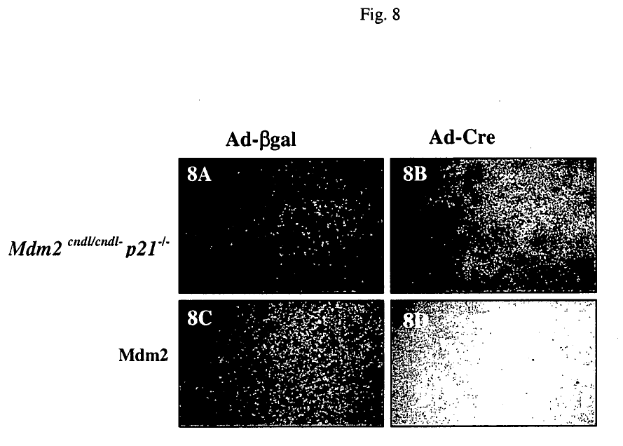

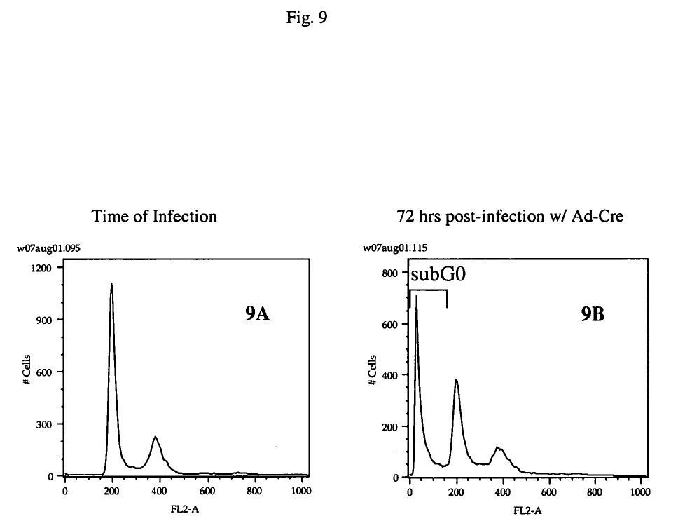

As is shown in FIG. 7, removing p21 from mdm2^{cndl/cndl} MEFs reduces the number of apoptotic cells by greater than 50% when compared to mdm2^{cndl/cndl} MEFs containing p21. Briefly, MEFs were prepared as described herein from mdm2^{cndl/cndl}-p21^{-/-} mice and transduced with either Ad-βgalactosidase virus or Ad-Cre virus. 48 hours later, the cells transduced with the Ad-Cre virus showed a much higher percentage of apoptotic cells than the control cells transduced with Ad-βgal virus. In FIG. 8, violet staining of cells derived from mdm2^{cndl/cndl}-p21^{-/-} or mdm2^{cndl/cndl} mice were infected with Ad-Cre (or Ad-βgal as a control); after 96 hours, it is clear that cells derived from mdm2^{cndl/cndl} mice undergo apoptosis by 96 hours post-infection with the Ad-Cre virus (FIG. 8B). However, cells lacking both Mdm2 and p21 survive (FIG. 8B), indicating that growth arrest may be required prior to the onset of apoptosis. As one theory, not meant to be limiting, p21 may be required for a fully executed apoptotic response.

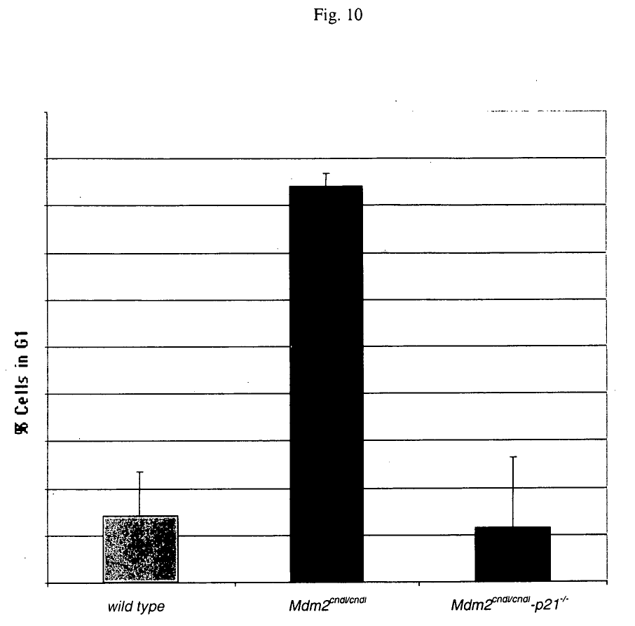

This evidence is contrary to the commonly accepted theory that removal of p21 facilitates apoptosis. In fact, as is shown in FIG. 10, mdm2^{cndl/cndl} or mdm2^{cndl/ml} MEFs accumulate more cells in G1 48 hours after viral transduction with Ad-Cre, unlike mdm2^{cndl/cndl}-p21^{-/-} MEFs that have similar numbers of cells found in G1 as compared to wildtype MEFs. Briefly, wild type or Mdm2^{cndl/cndl} or Mdm2^{cndl/cndl}-p21^{-/-} MEFs were plated at 5×10⁵ cells/100 cm plate. Twenty-four hours later, cells were counted and remaining plates were infected with 60,000 infectious particles of Ad-Cre in 5 mls of regular media (15%, GPS, DMEM as stated previously). 10 mls of media were added to plates 6 hours post-infection. Forty-eight hours following infection (triplicate plating), cells were trypsinized and fixed in 70% EtOH/PBS. Cells were then stained with propidium iodide for total DNA content (50 μg/ml) and analyzed via FACS analysis (our core facility does the analysis-I do not have more details than that). The results (shown in FIG. 10) indicate that for complete apoptosis to occur at 72-96 hours post-infection, cells must accumulate in G1 prior to apoptosis. Cells deficient in p21 do not arrest properly in G1; as one theory, not meant to be limiting, this failure to arrest properly may be the cause of the marked reduction of apoptotic cells at 72-96 hours post-infection.

### Example 7

### Inducible Suppression of Mdm2 Expression in the Epidermis

To evaluate the effect of suppression of mdm2 expression in the epidermis, mdm2^{cndl/cndl} mice were crossed with K5.CrePR1 mice (Zhou et al., Genesis 32:191-92 (2002); “K5 mice”). The K5 mice express a fusion protein consisting of Cre recombinase and a truncated progesterone receptor (CrePR1) targeted to the epidermis. The fusion protein is sequestered in the cytoplasm until a progesterone antagonist, such as RU486 (Mifepristone) which induces translocation of the fusion protein to the nucleus, where the Cre recombinase becomes active, and, in the K5-mdm2^{cndl/cndl} mice, excises exons 11 and 12 of the mdm2 gene, resulting in suppression of Mdm2 expression.

These K5-mdm2^{cndl/cndl} mice are useful for evaluating the effects of Mdm2 loss in the epidermis, for example, for screening agents that reverse or enhance the effects of Mdm2 loss. Such agents may be useful in treating conditions associated with aging, wound healing, or DNA damage.

### Example 8

### Inducible Suppression of Mdm2 Expression in MX1Cre Mice

To evaluate the effect of suppression of mdm2 expression in a temporally regulated manner, mdm2^{cndl/cndl} mice were crossed with MX1Cre mice. The MX1Cre mice express Cre recombinase under the control of the IFN-inducible MX1 promoter. (Schneider et al., Am. J. Renal Physiol. 284:F411-F417 (2003); Kahn et al., supra).

These MX1-mdm2^{cndl/cndl} mice are useful for evaluating the effect of loss of Mdm2 in a subset of tissues, that respond particularly well to IFN-gamma responses (in the liver, for example). For example, a response can be conditionally triggered to examine the global effects of Mdm2 in the adult mouse.

### Example 9

### Developmental Variability in Sensitivity to loss of Mdm2

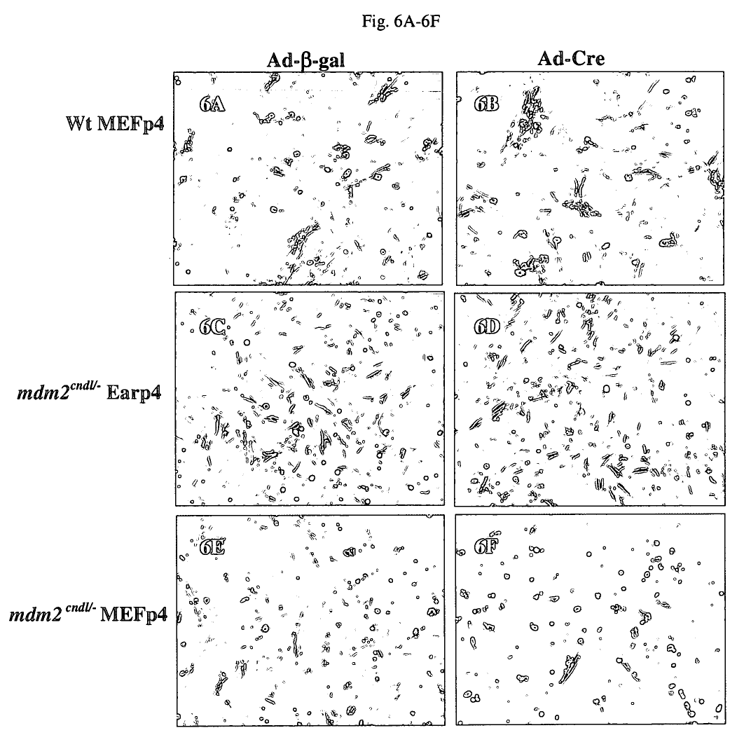

To evaluate whether there, were any differences in response to loss of Mdm2 in embryonic versus adult cells, fibroblasts were isolated from embryonic and adult wildtype or mdm2^{cndl/ml} mice and transduced with Ad-Cre or Ad-βgalactosidase virus. Briefly, MEFs harvested between e11.5 and e14.5 and adult somatic ear fibroblasts were processed as described herein. As is shown in FIG. 6, a much larger percentage of embryonic fibroblasts showed phenotypic evidence of apoptosis. Thus, adult somatic fibroblasts (cells removed from adult mice) respond differently to the loss of Mdm2 compared to embryonic fibroblasts.

The overall results of this experiment indicate that embryonic fibroblasts are sensitized to an apoptotic cascade when Mdm2 is removed. As one theory, not meant to be limiting, Mdm2 may be required for controlling p53 levels only in actively dividing cells (such as the MEFs) and this function may not be necessary in terminally differentiated or non-replicating cell types, such as the adult somatic fibroblasts.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An mdm2 gene targeting vector, comprising:
(a) a first targeting sequence substantially identical to a DNA sequence 5′ of one or more exons of the mdm2 gene;
(b) a first recombinase recognition sequence;
(c) a second targeting sequence substantially identical to a DNA sequence of one or more exons of the mdm2 gene;
(d) a second recombinase recognition sequence; and
(e) a third targeting sequence substantially identical to a DNA sequence 3′ of one or more exons of the mdm2 gene.

2. The targeting vector of any claim 1, further comprising one or more selection markers.

3. The targeting vector of claim 2, wherein the selection marker is an MC1-TK negative selection marker.

4. The targeting vector of claim 2, wherein the selection marker is an antibiotic resistance gene.

5. The targeting vector of claim 2, wherein the antibiotic resistance gene is a neomycin resistance gene.

6. The targeting vector of claim 5, wherein the neomycin resistance gene is PGK-Neo.

7. The targeting vector of claim 1, wherein the targeting sequence substantially identical to a DNA sequence of one or more exons of the mdm2 gene comprises the eleventh and/or twelfth exon's of the mdm2 gene.

8. The targeting vector of claim 1, wherein the recombinase recognition sequence is a lox sequence.

9. The targeting vector of claim 8, wherein the lox sequence is chosen from the group consisting of LoxP, Lox 66, Lox 71, Lox 511, Lox 512, and Lox 514, and variants thereof.

10. The targeting vector of claim 1, wherein the recombinase recognition sequence is a FLT sequence.

11. A method for producing a transgenic mouse comprising a conditional mdm2 allele, the method comprising:
transfecting a murine embryonic stem (ES) cell in vitro with the targeting vector of claim 1; and
generating a transgenic mouse from the ES cell.

12. An isolated cell transfected with the targeting vector of claim 1.

13. The isolated cell of claim 12, wherein the cell is an ES cell.

14. A transgenic non-human mammal whose somatic and germ cells comprise a conditional mdm2 allele having a first recombinase recognition sequence 5′ of at least one exon of an mdm2 gene and a second recombinase recognition sequence 3′ of at least one exon of the mdm2 sequence, wherein at least some cells of the mammal exhibit decreased mdm2 expression upon exposure to a recombinase.

15. The transgenic non-human mammal of claim 14, whose somatic and germ cells comprise two conditional mdm2 alleles having a first recombinase recognition sequence 5′ of at least one exon of the mdm2 gene and a second recombinase recognition sequence 3′ of at least one exon of the mdm2 sequence, wherein the animal exhibits decreased mdm2 expression upon exposure to Cre recombinase.

16. The transgenic non-human mammal of claim 14, wherein the recombinase is a Cre recombinase or a Flp recombinase.

17. The transgenic non-human mammal of claim 14, wherein the recombinase is the Cre recombinase of bacteriophage P1 or a variant thereof.

18. The transgenic non-human mammal of claim 14, wherein the recombinase is the Flp recombinase of Saccharomyces cerevisiae.

19. An isolated cell derived from the non-human mammal of claim 14.

20. The cell of claim 19, wherein the cell is a fibroblast.

21. The cell of claim 19, wherein the cell is a mouse embryonic fibroblast (MEF).

22. A method of evaluating the effect of suppression of Mdm2 function in a mammal or a cell, organ, or tissue derived from a mammal, the method comprising:
exposing the transgenic non-human mammal of claim 14, or a cell, organ, or tissue derived therefrom, to a recombinase under conditions sufficient to cause a decrease in Mdm2 expression; and
monitoring the mammal, cell, organ, or tissue for a response;
wherein the response indicates an effect of the suppression of Mdm2 .

23. The method of claim 22, wherein the response is associated with aging.

24. The method of claim 23, wherein the response associated with aging is selected from the group consisting of hair sparseness, reduced dermal thickness, reductions in bone density, lordokyphosis, lymphoid atrophy, increased body mass, decreased lifespan, increased organ mass, retarded wound healing, and increased subcutaneous adipose levels.

25. The method of claim 22, wherein the response is wound healing.

26. The method of claim 22, wherein the response is associated with cellular transformation.

27. The method of claim 25, wherein the response associated with cellular transformation is selected from the group consisting of cellular proliferation, tumor formation, tumor growth, and metastasis.

28. The method of claim 22, wherein the recombinase is administered to the mammal by a method selected from the group consisting of tail vein injection, oral administration, and nasal administration.

29. The method of claim 22, further comprising administering a test agent to the mammal, cell, tissue or organ, and monitoring the mammal, cell, tissue or organ for a response in the presence of the test agent.


SEQUENCE LISTING (12 sequences)

SEQ ID NO: 1 - DNA, 2167 bp, Mus musculus
gtagtgcttgggtttaagaaattatcttactttttaatatggaagtgtcttgtctgccagaatgtacaccacatgcatgcagtgctttgaggccacaggagggtgtggagtcccctggaactggggttagggatggttggagccactgtgtgggtgccaggaaagtgggttgactaccacatctctgccctagttttgagtgctgcagattaaatccagggctctgtgcgcactgggcaagtgttctaccagttaattacatccccaacacccaacagtcagtgagccaacctctctgcggatctctgcctatctagtttcttagaatagaacctgacctgctgtcaaaacagaatatatatatatatatatatatatatatatatatatatatatatatatatgctcttctaaaaatcttccatattaaaaatttgttactttttaaacttaagtgaaaatgcttttaatacaactgtgttttgctttgttttaacttacttatgagaacattagtctcagtagttttaccctttatttacagggaacactatgcctgatagtgtttcaaataatttgttaaaggatcctacaactaattattacatttaataagacaggctagaacttgaaagctcagcggttaagagccctgactgctcttccagaggtcctgagttcaactcccagcaaccacatggtggctcacaaccatctataatgggatctggtgccctcttctggtgtgtctgaagacagcaacagtatacttagatataataataaattaatcttaaaaaaaaaagactacttttatgtcaagctgactgtattttctggaagagaaatgatgtgtctgcagttaagcagcatcttggtagcaaattgagaatgcctgtgtcctgaattctagagcagtgtatttttatcagttgtaagtaagttgatttgcggatgaacttagtgtatgatttgaagttgaatctcttgtttgcatttttgtgagacagggtctcttatttagttctgactcttggctgtcctggaactcactgtatagaccaggctggttttgaacctacagagatcttccagtctctgcctcctgaatgaatgctaggattaaaggcatacaccacatgcctggcctgaagttgaatcttttaatctcagcactcaggaagcagggacagcagagctctctgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggtaatagccaatatagtgtcacatgaggccccaggccaactaaggctgcatcggaaaacccagatgccagcttggtgtttgtgtgtcgtcactagcactccgtcacactagcctgtgagtcagggaggccttcgtcttcctgtgtttaataggataaactcacagtggtgcttcagataatagtgggtgtgcatataatcacacacgtaggaaaggtcattgtggaagatctttttatggtattgtgggaaccttaaccaaagaaagatgagtggggcttagaaactcagattatcttactgagaaaaagttagtgtgtggattgatttaggaaacaagatgtggctggagcatgggtattgaatagggtggagggtgtgtttagctggacatttgaaaatgttaatgcagtcattctgagtgtaacctcggggctaaggggctctgacttttccttcatgaggtaggacttttgagtaaggtagtaaccttgaacccagtctatgtggtctggggctgagaaggaaatagtggctcgggatgaacccgggagctctaaatgaggcccagaggcaatgcatttacattcgctgcattcttcctccatag

SEQ ID NO: 2 - DNA, 156 bp, Mus musculus
gatcttgacgatggcgtaagtgagcattctggtgattgcctggatcaggattcagtttctgatcagtttagcgtggaatttgaagttgagtctctggactcggaagattacagcctgagtgacgaagggcacgagctctcagatgaggatgatgag

SEQ ID NO: 3 - DNA, 1357 bp, Mus musculus
gtgggtttctgtgtctgtcagccttttagtaagatgaagaggccatctattcagagtctctggaaagtcttgctttttcatttgttcaagatagaatattgctttgtggatttgaggtgaaattttatagtttctttgcttgatttatttatttagatttatttgttatgtatacagcattctgcctgcatgtatgtctgcaggccagaagagggcactagatatggttactagccaccatgtggttgctgggaattgaactcaggacctttggaagaacagacagtgctcttaacctctgagtcatctctccagccctttgcttgatttattatagtctcacattactaaataaaaaacttagaggagagtttgaacaatgtttttaaactttttttgagttgatggggtgtttttgttttgtttgttttgctttttgagacttggttttttttttttgtttttgtttttgtttttgtttttttttttggtttttcgagacagggtttctctgtgtagccgtggctgtcctggaactcactttgtagaccaggctggcctcgaactcagaaatccacctgcctctgcctcccaagtgctgggattaaaggccttcgccaccacgcccggcgagacttggtttttctgtatggccctagctgtcctggaacttgctttgtagaccaggctggcctcagatctgcctgcctttgcttcccaagtcctggaattaaaggtgtgcgccaccactgctctgctaatgatggatgaacattctcataatggctggaccacaaagttctccatttggtcttcccatttatgtatgtatgtgtgtgtgtgtatttgtttttgagacagggtaccaccgtgtatccctggctgaccctgaactggctatctagaccagcctggcctcaaactcatgggaatcctcttgcctctgcctcctgattgctgggatgaaaggtgtgagcccctacttctgcccctcccttctcagtactgatttgtttttcaccaatgtaaagcatgcaacgaacagtgtccaataggacccgagtatctctttctcgggagcaactctacatttctagttgttttctgtctaataactctaaatttataaaatgtcttcttgagtgttttgtttggttttaagtatctaacaactcttgccattgcgagtaaaggttttcctatcctgcgtttcctgcttgccttgaactgcatttaactttctttcagaactcttcgtccacagttaatggctcaagctgtttgttttaagacttactgagaattctggcttcttggttgaagggttgaattgatgctaatgaatttgtcttactag

SEQ ID NO: 4 - DNA, 97 bp, Mus musculus
gtctatcgggtcacagtctatcagacaggagaaagcgatacagactcttttgaaggagatcctgagatttccttagctgtaagtatatagattcttc

SEQ ID NO: 5 - DNA, 340 bp, Mus musculus
aaagaaataaaacacaatgttgagatcagaattaggactataggtagttgtgagccaccacgtgtgtgctcagaacctgagccatctctagcctgtcactatttcatataggtcaccttttgtgtgtgtgtgtgtgtgtaggggaaagtacctagagtctactctattagctaatttctggtatatagtattagatatattcctcatgttctttgcctatttatgtaaaatatgaaatgctgaaaattaagctacatggtatacttttaccttagccacattgaagttattagccttgctatgaacactgactgaagagttactgtcttttttattgaag

SEQ ID NO: 6 - DNA, 576 bp, Mus musculus
gactattggaagtgtacctcatgcaatgaaatgaatcctccccttccatcacactgcaaaagatgctggacccttcgtgagaactggcttccagacgataaggggaaagataaagtggaaatctctgaaaaagccaaactggaaaactcagctcaggcagaagaaggcttggatgtgcctgatggcaaaaagctgacagagaatgatgctaaagagccatgtgctgaggaggacagcgaggagaaggccgaacagacgcccctgtcccaggagagtgacgactattcccaaccatcgacttccagcagcattgtttatagcagccaagaaagcgtgaaagagttgaaggaggaaacgcaggacaaagacgagagtgtggaatctagcttctccctgaatgccatcgaaccatgtgtgatctgccaggggcggcctaaaaatggctgcattgttcacggcaagactggacacctcatgtcatgtttcacgtgtgcaaagaagctaaaaaaaagaaacaagccctgcccagtgtgcagacagccaatccaaatgattgtgctaacttacttcaactag

SEQ ID NO: 7 - DNA, 3044 bp, Mus musculus
ctgacctgctcacaaaaatagaattttatatttctaactatatgacccccaaattagacaacatgggtattatttttatacattaaagccagaaaactgtcttagtccacataaaattcacttataatttatcctggagagtaaatatggtgaatattttcttcctttttagggaaatttcacttgtttattttatatttgtgttttaagtaatttgcattggctgtttatattttccttatattttaaataatctccgcttggaaggactttggaagtgtatgtgagaagtcctttccatctcctgcagatgatggtggaccttcctcatcaagggctacagaagtacttgatttctgttttttttgttaataataagaacatttaatttatttagtgtctttcatgtaaagagttaaagactatgtgaaggattgtatatttaagttattgaaattctgaaactgtagtaatctaaaatgtgtgagttgtgggctgcagagaagactcagccagtaaaggcacctgctgtgtacacccactgacacacatttgatccttggaacccccaggaagagtgaaccagttccacaaagttgttccctgatcttcaaatggatgcacgcacgcacgcacacacacgcgcgcgtgcgcacacacacacacacacacacacacacacacacacacagttttaaaggcatgcattgcatctggtggtatgtaagtgaaaacacacgccttattttccagcattttcagctttttgtcataggggtgtggcacaagtgttgcagtttgtcccaggttgaaaagcctgaggctggtagaagcgcctttttgcctcagctccgtggttcctggtggttgcctatgtttcaggcctgtacttaggctaggtttagaaaccagcccattcagaaagactgaatcagaacatggataaagtgaactcattctaagatgactcgtctatccatgtagattaatctcctggttcataataggcctcttccctttgattgaagggtcacgtctaagtatagaaaacataaaactgtaaggtagaggaagcgaaggatagctttgtattaatgttgcgttaaagcttcagagacaagaacaagaacactcctcccacgtgacagcatttgaataggaggcggtgggtgcggcagcctgggcagcttcagtcccgatttacaataaagtaccttgtgtgttattagttcttaaatgtttatttagaaatggcattgatgttatttatttgcaaataaatggtttattgaagaattgtgaaagagatttgtcttacacatttttttgtcagtgattttgtttttaaagatttgttcattatatgtgagtacactgtagctgtcttcagacgcaccagaagagggcgtcagatctcattacagatggttgtgacccaccatgtggtggctgggagttgaactcaggacctttggaagagcagtcagtgctcttaaccaccgagccatctcagcagcccccatcagtgatctttttataattcatttttattttatgttcattggtgttatggctatatgtctgtgagggtgacagaaaccctggaactggagttacagacaggtgtgagctgccctgtgggtgctgggaaattgatccattgctctccacctttttaaaatttgtgtgtatgaataattcgtccacatgtatatgtattgtgtgcatacactgccttgaactacatgcatgcagtgcctgaagaggccggaagagggcatcagatccttaggacccgagttccaggctaggagcctaaccctgtcctctgtaagagcagatgctgctcttgaccacacagctgtctacatgccgccttttttttttttttttaaggtttatatattttatgtatatgagtacactgtagctgtacagatggttgtgagccttcatatggttgttgggaattgaattttaggacctctgctggctgcttgctccggctcaaagctttatttattataagtacactgtagctgtcttactataagagggcatcaagtctcactacaggtggttgtgagccaccatgtagttgctgggatttgaactcagaatttctgggagagcagccagtgttcttaaccactcagctatctctccagcccctaatagctacaaatttaaatactttaggcaattttgcctagggatttcccaatatattatattgcaatggattttcttttgagtcggagaaatgtttgcgatttaacttaaagttaagatacttgtgtggaggagatggctcagtgcttgtcttgtgccctgattccatccccagaaaccgtggatatggtggcactcatctgtaaccacagcatgagagatgacaggcagacccacaggcgaatcatctggaagcttgctggtcagctggcttgcagtatgcatcatgacagaaacaagaggctaccacaaggtggaaggccggagactgttaggttttgggtgttgctaaatactggccctggttgcctccaggggctgggaaattctcacttaaacccagatgatgctctgtaatcttgtcccccaagttatccccgattggcgaataaagatgcctgcagcctgtagctgtgtggaagagagacaggtggggtttgggggtctgaggcaggaagaagaaagaggtggagagaggagagtgtaggtgagtctggaagaagtcagtgctcttaatccctgagccatctttctagccccatatagcagtcttttctagtttgtagatttttgttatgtttgcaaacaaaacagtagtggaaagagacaacattcactatatgaatattctttattgataatcagataatgtaaataaaataagctcagaggtagtcatggtccctcccaagcaaacgttgtaatcttagataatctgagaatttatttatttttctgtgtagccctggctgtcttggaactcactgtgtagaccaggcaggctggatttgaactcagagtcccctctgcct

SEQ ID NO: 8 - DNA, 81 bp, phage
gcttgggctgcaggtcgagggacctaataacttcgtatagcatacattatacgaagttatattaagggttccggatcccgg

SEQ ID NO: 9 - DNA, 24 bp, artificial
  PCR primer sequence based on m. musculus sequence of mdm2
gcttactatgagttccaagtgcag

SEQ ID NO: 10 - DNA, 21 bp, artificial
  PCR primer based on M. musculus sequence of mdm2
cctggactagccatcttagaa

SEQ ID NO: 11 - DNA, 26 bp, artificial
  PCR primer based on M. musculus sequence of mdm2
atgtgcaataccaacatgtctgtgtc

SEQ ID NO: 12 - DNA, 25 bp, artificial
  PCR primer based on M. musculus sequence of mdm2
gcagatcacacatggttcgatggca